(19) United States
(12) Patent Application Publication
(10) Pub. No.: US 2023/0301899 A1
(43) Pub. Date: Sep. 28, 2023

(54) EXTRACT OF SPIRODELA POLYRHIZA AND ITS COSMETIC USES

(71) Applicant: SOCIETE INDUSTRIELLE LIMOUSINE D'APPLICATION BIOLOGIQUE, Objat (FR)

(72) Inventor: Jean PAUFIQUE, OBJAT (FR)

(21) Appl. No.: 18/125,858

(22) Filed: Mar. 24, 2023

(30) Foreign Application Priority Data
Mar. 25, 2022 (FR) 2202694

(51) Int. Cl. A61K 8/9794; A61K 8/73; A61Q 19/00; A61Q 19/08

(52) U.S. Cl. CPC A61K 8/9794; A61K 8/73; A61Q 19/007; A61Q 19/08

(57) ABSTRACT

The present invention relates to a cosmetic active substance obtained from a duckweed of the species Spirodela polyrhiza and its cosmetic uses, in particular its use as a plumping agent.

### TECHNICAL FIELD

The invention relates to a cosmetic active substance obtained from a duckweed of the species Spirodela polyrhiza and its cosmetic uses, in particular its use as a plumping agent.

### PRIOR ART

The cosmetics industry is interested in problems related to dehydration, in particular cutaneous dehydration. Dehydrated skins are characterized by a water deficit, the causes of which are multifactorial. Among the most frequent factors, mention may in particular be made of environmental factors, hormonal factors, or else those associated with taking medication. According to preconceived notions, dehydrated skin is dry skin. However, all types of skin may be affected, whether the skin is dry, mixed or greasy. A sensation of tugging, lack of flexibility, loss of volume, a dull complexion, wrinkles and fine lines that appear are just some of the symptoms of dehydrated skin.

There is therefore a need for novel cosmetic active substances having a high hygroscopic potential allowing the effect of plumping, hydration and boosting the complexion radiance of the skin.

To meet this need, the inventor has focused on natural raw materials to develop ingredients having a high hygroscopic potential. Particular attention has been paid to macromolecules of the pectin type derived from the aquatic world and specifically to those derived from duckweed.

Used for more than 2000 years in traditional Chinese medicine for its abilities to promote water metabolism, duckweed has a plant wall comprising original pectins, thus helping to retain water in plant cells of the plant. These original-structure pectins, apiogalacturonans (APG), have been identified only in some aquatic species, such as in the species Lemna minor, Zostera marina, which are also known in cosmetics for their antioxidant effects.

In this context, the inventor interested in a particular duckweed species, the species Spirodela polyrhiza, and has discovered its higher hygroscopic potential than other glycocompounds of pectic (homogalacturonans) or non-pectic (fructosans) origin and its remarkable moisturizing and repulsive properties.

### SUMMARY OF THE INVENTION

Thus, the invention relates to a new cosmetic active substance obtained from a duckweed belonging to the species Spirodela polyrhiza comprising apiogalacturonans, in particular its use as a plumping agent. Preferentially, the active substance according to the invention is enriched with apiogalacturonans.

Duckweed Spirodela polyrhiza also referred to as Lemna polyrhiza, or Lemna major or Spirodela polyrrhiza belonging to the family of the Lemnaceae, is a perennial aquatic plant that float atop the surface of ponds, thus making it possible to regulate temperature variations between the air and the water. It consists of a frond (or thallus) that can reach 10 mm in diameter, filled with case of air pockets allowing it to float, often having 5 to 12 ribs, and comprising from 2 to 16 fasciated roots not exceeding 3 cm long.

Duckweed Spirodela polyrhiza has an original pectic wall containing apiogalacturonans (APG). Their role is to ensure water exchanges and to confer high plasticity to the plant during its multiplication. In their native form, apiogalacturonans from Spirodela polyrhiza have a linear structure composed of a linear chain of galacturonic acids formed by strong bonds, and branches of di-apiose units branched by weak bonds that are extremely fragile. Depolluting, antioxidant, depigmenting, anti-wrinkle effects, and effects of stimulating the growth of hair, eyelashes or eyebrows have previously been reported for duckweed extracts.

Thus, the invention relates to an active substance comprising at least one extract of Spirodela polyrhiza rich in apiogalacturonans, as well as its cosmetic use preferentially as a plumping and/or moisturizing and/or for improving the radiance of the complexion.

In order to develop such a naturally derived ingredient (per the definitions of the ISO 16128 standard) that is enriched with apiogalacturonans while preserving their native structures, in particular the di-apiose units, the extract included in the active substance according to the invention is preferentially a hydrolysate. This can be carried out by chemical hydrolysis, or by chemical hydrolysis and enzymatic hydrolysis.

Advantageously, the active substance according to the invention has a hygroscopic potential greater than or equal to 15 molecules of water interacting per monomer.

The extract according to the invention was characterized and the extract comprises carbohydrates, commonly called sugars. Preferentially, these represent at least 70% by weight of the dry matter of the extract.

The carbohydrates included in the extract according to the invention particularly comprise apiogalacturonans, which preferentially represent at least 40% by weight of the dry matter of the extract, more preferentially between 40 and 70% by weight of the dry matter of the extract.

Thus, the extract according to the invention comprises carbohydrates composed of apiogalacturonans and optionally other sugars. These other sugars preferentially represent between 20 and 35% by weight of the dry matter of the extract. By way of example, the other sugars may be derived from rhamnogalacturonans-I (RG-I).

The apiogalacturonans present in the extract are composed of a linear chain of branched galacturonic acids of apiose units, more preferentially of di-apiose units. The extract according to the invention preferentially comprises linked apioses.

According to a preferred object of the invention, the extract has a galacturonic acid/apiose ratio of between 1 and 5, preferentially between 2 and 4.

According to another object, the apiogalacturonans have a molar mass of between 180 Da and 1360 kDa, as well as a mean molar mass of 38 kDa. Preferentially at least 40% of the apiogalacturonans have a molar mass of between 3600 Da and 200 kDa.

According to a particularly preferred object, the extract is likely to be obtained by a method comprising the following steps:
a. Solubilization of at least 50 g/L of Spirodela polyrhiza in water,
b. Chemical hydrolysis of acid nature, or chemical hydrolysis of acid nature and enzymatic hydrolysis,
c. Separation of the soluble and insoluble phases and recovery of the soluble phase,
d. Heat treatment
e. Purification and concentration of the soluble phase,
f. Filtration and sterilizing filtration.

The invention also relates to a method for preparing the active substance according to the invention, comprising in particular a solubilization of a biomass of Spirodela polyrhiza in water, chemical hydrolysis of acid nature, or chemical hydrolysis and enzymatic hydrolysis, separation of the soluble and insoluble phase, then recovery of the soluble phase, purification and optionally concentration of the soluble phase followed by filtration and sterilizing filtration.

The active substance according to the invention advantageously has an effect selected from a plumping effect, a radiance booster effect, that is to say improving the radiance of the complexion, and a moisturizing effect, thus making it possible to address the problems of the prior art. Also, it can be used for cosmetic applications and thus be integrated into a cosmetic composition, advantageously a composition in a form suitable for topical application.

Thus, according to another aspect, the invention relates to a cosmetic composition for topical application comprising at least one active substance according to any of the above-described embodiments and a physiologically acceptable medium.

Preferentially, the active substance contained in the composition according to the invention represents at least 0.1% by weight of the total weight of the composition.

The active substance or the composition according to the invention is intended for cosmetic applications. Thus, according to another aspect, the invention relates to the cosmetic use of the active substance according to any of the embodiments or a composition according to any of the embodiments. Preferentially, the cosmetic use targets a topical application making it possible to obtain a plumping and/or moisturizing and/or complexion radiance-improving effect.

In particular, the active substance or the composition according to the invention increases the skin cell volume, preferentially the volume of the keratinocytes, and/or the expression of aquaporin 3 and/or the synthesis of the BGT-1 transporter.

Finally, according to a last aspect, the invention also relates to a method for cosmetic treatment of dehydrated skin which consists of applying to the dehydrated skin a composition according to the invention or an active substance as described above.

Other features and advantages will emerge from the detailed description of the invention and the following examples and figures.

### BRIEF DESCRIPTION OF THE FIGURES

FIG. 1 shows the effect of the active substance according to the invention, extracts outside the invention according to Examples 3 and 4, on the amount, depth and maintenance over time of the water D2O captured by the skin, 3 hours after a single application.

FIG. 2 shows the effect of the active substance according to the invention on the total amount of water and the depth at the epidermis of volunteers 3 hours after a single application.

FIG. 3 shows the effect of the active substance according to the invention on the total amount of water at the epidermis of Caucasian volunteers 42 days after a twice-daily application.

FIG. 4 shows the effect of the active substance according to the invention on the radiance of the complexion after 21 and 42 days of twice-daily treatment by Caucasian volunteers.

FIG. 5 shows the effect of the active substance according to the invention on the radiance of the complexion after 21 and 42 days of twice-daily treatment by Asian volunteers.

### DETAILED DESCRIPTION OF THE INVENTION

### Definitions

Within the meaning of the invention, “cosmetic active substance” means an extract comprising at least one molecule, preferentially a set of molecules having a cosmetic effect on the skin, when applied topically.

Within the meaning of the invention, “naturally derived ingredient” means a cosmetic ingredient obtained by chemical and/or biological methods defined with the aim of chemically modifying it. These chemical modifications are intentional, and the structures of the molecules obtained are therefore no longer identical to the molecules present in nature.

Within the meaning of the invention, “hydrolysate of Spirodela polyrhiza” means an active substance derived from duckweed of the species Spirodela polyrhiza, obtained by a method comprising at least one step of hydrolysis of Spirodela polyrhiza. The term Spirodela polyrhiza excludes molecules produced solely by fermentation of Spirodela polyrhiza by a microorganism or an infusion/decoction/maceration of Spirodela polyrhiza.

Within the meaning of the invention, “Spirodela polyrhiza” means duckweed of the family of the Lemnaceae family and the species Spirodela polyrhiza also known under the names Lemna polyrhiza, Lemna major, Lemna maxima, Spirodela polyrrhiza, thus excluding the species Lemna minor or Zostera marina.

Within the meaning of the invention, “plumping” means an effect making it possible to increase the skin cell volume. This effect is often associated with the term “rejuvenated skin”.

Within the meaning of the invention, “hygroscopic potential of a molecule” is understood to mean the capacity of the structure of a molecule to interact, to bind and to retain water molecules around its chemical structure.

Within the meaning of the invention, “dehydrated skin” is intended to mean a skin suffering from a water deficit, thus having a sensation of discomfort, shaving, or a lack of flexibility. These signs of dehydration are not those of dry skin, which suffers from a lack of fat. Dehydrated skin affects all skin types whether it is dry, mixed, or greasy.

Within the meaning of the invention, “monomer” means a monosaccharide of galacturonic acid or a monosaccharide of apiose.

Within the meaning of the invention, “galacturonic acid/apiose ratio” means the ratio between the number of moles of galacturonic acid and the number of moles of apiose in the apiogalacturonans.

Active Substance According to the Invention

The subject of the present invention is therefore a cosmetic active substance comprising at least one extract of Spirodela polyrhiza, the extract comprising apiogalacturonans. Such an active substance is particularly of interest and makes it possible to address the drawbacks of the prior art in that it has a high hygroscopic potential obtained by the preservation of the apiogalacturonan molecules included in the extract of Spirodela polyrhiza. Thanks to its remarkable hygroscopic properties, the active substance according to the invention moisturizes the skin from the stratum corneum to the upper dermis, resulting in a plumping, moisturizing or radiance-boosting effect.

Also, the active substance according to the invention preferentially has a hygroscopic potential greater than or equal to 15 molecules of water in interaction per monomer, more preferentially greater than or equal to 20 molecules of water in interaction per monomer.

The active substance according to the invention is enriched with apiogalacturonans, preferentially apiogalacturonans representing at least 40% by weight of the dry matter of the extract; more preferentially they represent between 40% and 70% by weight of the dry matter of the extract.

The active substance according to the invention comprises sugars, in particular carbohydrates (neutral sugars and uronic acids); carbohydrates preferentially represent at least 70% by weight of the dry matter of the extract. The content of total neutral sugars in said active substance can be determined by the DUBOIS method (Dubois M. et al., Analytical chemistry, 28, 3, 350-356, 1956). The content of total neutral sugars in the active substance according to the invention is expressed as a percentage relative to dry matter.

The carbohydrates present in the extract can be in the majority of polysaccharides, oligosaccharides and very few monosaccharides. Thus, the carbohydrates of the active substance according to the invention have an average molar mass of 38 kDa, and molar masses of between 180 Da and 1360 kDa.

In particular, the carbohydrates present in the extract according to the invention comprise apiogalacturonans and other sugars. Preferentially, the other sugars represent between 20 and 35% by weight of the dry matter of the extract. The other sugars may for example comprise rhamnose, galactose, derived from rhamnogalacturonan-I (RG-I), arabinose or glucose.

The apiogalacturonans present in the extract are preferentially composed of a linear chain of galacturonic acids connected from branches of di-apiose units.

The cosmetic active substance according to the invention can be in liquid form, in solid form or in film form.

When it is in liquid form, the active substance according to the invention is exclusively composed of Spirodela polyrhiza extract accompanied by stabilizers and/or storage systems.

The extract in liquid form is preferentially in the form of a clear liquid aqueous solution, with a low odor and a very pale yellow color with light yellow. However, it may be more colored and/or be discolored by any method known to a person skilled in the art.

The inventor also has determined the dry matter content of the extract. This can be determined by weighing the residues resulting from the drying of the extract according to the invention at 105° C. in an oven until a constant weight is obtained. Preferentially, the extract according to the invention in liquid form has a dry matter content of 3 g/L to 20 g/L, preferentially 5 g/L to 8 g/L.

When it is in solid form, the active substance according to the invention is preferentially composed of Spirodela polyrhiza as previously described and by a support selected from maltodextrin, gum arabic, soybean lecithin or isomalt. According to one particularly suitable embodiment, the extract represents at least 10% by weight of the active substance and the support at most 90% by weight of the active substance.

In the case of a solid form wherein the active substance is associated with a support, the protein contents, sugars and ash in the active substance are modified, the support generally consisting of sugars.

The active substance according to the invention can also be presented in the form of a film. In this case, the extract of Spirodela polyrhiza preferentially represents at least 0.1% by weight of the film.

When it is in the form of a film, the active substance comprises:
at least the extract of Spirodela polyrhiza according to the invention.
at least one mineral filler, and
at least one polymer of natural origin, and
at least one plasticizer, and
at least one surfactant, and

The polymer of natural origin may be chosen from: Pectin, tamarind gum, alginate, pullulan, psyllium, xanthan, guar, tara, carob, agar, gum arabic, gellan, dextran, carrageenan, cellulose, konjac and chitosan.

The plasticizer may be chosen from: Glycerol, sorbitol, saccharose, erythritol, urea, propylene glycol and butylene glycol.

The mineral filler may be chosen from: Calcium carbonate, green clay, kaolin, perlite, talc, magnesium silicate, mica, diatomaceous sericite, silica, calcium sulfate, calcium chloride, potassium chloride, iron oxide and zinc oxide.

The active substance may also comprise a pigment for coloring the film.

The extract of Spirodela polyrhiza is preferentially a hydrolysate. The hydrolysate can be obtained by any type of hydrolysis, preferentially by chemical hydrolysis of acid nature, or by chemical hydrolysis of acid nature and enzymatic hydrolysis.

Also, the extract according to the invention is preferentially an acid hydrolysate or an acid-enzymatic hydrolysate.

According to a particularly preferred embodiment, the extract of the cosmetic active substance according to the invention is capable of being obtained by an extraction method comprising the following steps:
a. Solubilization of at least 50 g/L of Spirodela polyrhiza in water,
b. Chemical hydrolysis of acid nature, or chemical hydrolysis and enzymatic hydrolysis,
c. Separation of the soluble and insoluble phases and recovery of the soluble phase,
d. Heat treatment,
e. Purification and concentration of the soluble phase,
f. Filtration and sterilizing filtration.

Cosmetic Composition According to the Invention

The active substance according to the invention may optionally be integrated into a cosmetic composition, in particular a composition comprising at least 0.1% by weight of said active substance and a physiologically acceptable medium, preferentially a cosmetically acceptable medium.

These compositions may in particular be in the form of oil-in-water emulsions, water-in-oil emulsions, multiple emulsions (Water/Oil/Water or Oil/Water/Oil) which may optionally be microemulsions or nanoemulsions, or in the form of solutions, suspensions, hydrodispersions, aqueous gels, powders, or foundation or in the form of a film. They may be more or less fluid and have the appearance of creams, emulsions, gels, masks or any other aspects of healthy skin care cosmetics.

These may be compositions comprising at least 0.1% of the liquid active substance according to the invention, preferentially between 0.1 and 10%.

These compositions comprise, in addition to the active substance, a physiologically acceptable medium such as a cosmetically acceptable medium, that is to say which does not cause sensations of discomfort for the user, such as redness, tugging or tingling.

As an additive, the compositions according to the invention may contain at least one compound selected from:
oils, which may be chosen in particular from linear or cyclic, volatile or non-volatile silicone oils,
waxes, such as ozokerite, polyethylene wax, beeswax or carnauba wax,
silicone elastomers,
surfactants, preferably emulsifiers, whether non-ionic, anionic, cationic or am-photeric,
co-surfactants, such as linear fatty alcohols,
thickeners and/or gelling agents,
humectants, such as polyols such as glycerin,
dyes, preservatives, fillers,
tensors,
sequestrants,
perfumes,
and mixtures thereof, without this list being exhaustive.

Examples of such additives are cited in particular in the CTFA Dictionary (International Cosmetic Ingredient Dictionary and Handbook published by the Personal Care Product Council).

Of course, a person skilled in the art will take care to choose any additional, active or non-active compounds, and their quantity, such that the advantageous properties of the mixture are not, or substantially not, altered by the envisaged additive.

Method for Extracting an Extract According to the Invention

The extract constituting or contained in the active substance according to the invention can be obtained by any means.

According to a preferred embodiment, the extraction method comprises at least one step of extraction of duckweed Spirodela polyrhiza, such as a hydrolysis step selected from chemical hydrolysis of acid nature, enzymatic hydrolysis and combination thereof.

Prior to the method for obtaining the extract as such, culturing the biomass of Spirodela polyrhiza is carried out in a medium suitable for their development, for example a suitable culture medium, in a conventional manner for a person skilled in the art. Once the biomass has been obtained, an extraction step is carried out, preferentially an acid hydrolysis, or acid and enzymatic hydrolyses, in order to obtain the active molecules.

According to a particularly suitable embodiment, the active substance according to the invention is obtained by implementing the following steps:
a. Solubilization of at least 50 g/L of Spirodela polyrhiza in water,
b. Chemical hydrolysis of acid nature, or chemical hydrolysis of acid nature and enzymatic hydrolysis,
c. Separation of the soluble and insoluble phases and recovery of the soluble phase,
d. Purification and concentration of the soluble phase,
e. Filtration and sterilizing filtration.

The hydrolysis conditions are selected to obtain an extract enriched with apiogalacturonans, preserving a galacturonic acid structure and di-apiose branches.

The separation of the soluble and insoluble phase is carried out by any means known to a person skilled in the art, for example by centrifugation, filtration or decantation. The separation of the soluble and insoluble phases is carried out in order to recover the soluble phase containing, inter alia, soluble sugars, such as apiogalacturonans.

Optionally, an additional heat treatment step can be carried out to inactivate the enzyme used. This inactivation is then carried out according to the enzyme supplier's recommendations.

Optionally, the process comprises a filtration step after the recovery of the soluble phase in order to remove the particles still in suspension. Thus, this filtration step allows the purification of the soluble phase recovered and is carried out in order to select the active molecules.

The hydrolysate obtained at this stage may optionally be further concentrated and/or purified, preferentially by successive ultrafiltration steps through membranes of different porosity, while retaining the active molecules at each step and/or by a chromatographic method.

The hydrolysate obtained after hydrolysis and filtration, before or after concentration and sterilizing filtration, is a hydrolysate of Spirodela polyrhiza, and constitutes a first form of the active substance according to the invention, being in liquid form at that time.

The hydrolysate obtained can then be dried and associated or not with a support, in order to be in solid form. This phase can be carried out by implementing the following steps:
an atomization support, preferably maltodextrin, is added to the hydrolysate of Spirodela polyrhiza, at most 90% (by mass/volume);
this solution is then concentrated under vacuum;
any bacteria that may be present are removed by heat treatment;
atomization makes it possible to obtain a powder.

The steps of the methods described above, taken individually are common in the field of extraction of active substances from natural raw materials and the person skilled in the art is able to adjust the reaction parameters on the basis of his general knowledge.

Cosmetic Use

The active substance according to the invention or the composition according to the invention is intended to be used on healthy skin, particularly dehydrated healthy skin, in particular as a plumping, moisturizing, and radiance-boosting agent.

Preferentially, the cosmetic use of the active substance according to the invention or of the composition according to the invention relates to a plumping and/or moisturizing effect and/or to improve the radiance of the complexion, more preferentially to increase the skin cell volume, and/or the expression of aquaporin 3 and/or the synthesis of the BGT-1 transporter and/or the natural moisturizing factors (NMF).

Aquaporins, small channels integrated into cell membranes, allow the dynamic and effective circulation of water. Aquaporin 3 is predominantly present in the skin, very particularly in the membrane of keratinocytes. The optimal distribution of these channels in all the layers depends on the level of hydration of the epidermis. By ensuring the flow of three billion molecules of water per second, they guarantee a correctly irrigated epidermis all the way to the surface.

The osmolytic transporter BGT-1 (carrier-GABA transport) is a regulator of the intracellular content of betaine, one of the majority osmolytes of the epidermis. Indeed, one of the defense mechanisms of the cells against water stress is osmotic adjustment. Osmolytes are small soluble molecules that play a role in protecting the cell against water deficit. Under stress conditions, osmolytes accumulate within the cell by virtue of specific transporters activity in order to avoid water loss and to maintain cellular structures. In addition to combating the dehydration of the cells, betaine plays a role of chaperone which stabilizes the structure and the function of the proteins, in particular those involved in tight junctions.

Natural moisturizing factors (NMFs) group together a set of intracorneocytic hydrophilic substances contained at the level of the stratum corneum, derived mainly from the degradation of filaggrin, and consist essentially of amino acids (40%), the majority of which is serne, of PCA (pyrrolidone-carboxylic acid) (12%), of lactate (12%), of urea (7%) and of inorganic ions. Their presence makes it possible to capture the free water, to retain it within the corneocytes and thus to play an important role in maintaining the physical properties of the stratum corneum. Lactate in particular will maintain the acidic pH of the stratum corneum, one of the important parameters guaranteeing the integrity and cohesion of the stratum corneum.

The active substance according to the invention is particularly of interest for the skin plumping, moisturizing, and radiance-boosting effects thereof.

The invention therefore also relates to a cosmetic method for treating the skin for a plumping and/or moisturizing effect and/or to improve the radiance of the complexion, which consists of the topical application to the dehydrated healthy skin of a person of such an active substance according to the invention or of such a composition according to the invention.

The active substance according to the invention or the composition according to the invention thus makes it possible to improve the plumping effect and moisturization. Indeed, in volunteers having dehydrated skin at the face and a dull complexion, after 42 days of twice-daily applications of a cosmetic composition containing the active substance, the active substance significantly plumps the skin of the face. This effect is accompanied by smoothing the cutaneous microrelief and improving the characteristic parameters of the radiance of the complexion of the volunteers.

The invention is now illustrated by non-limiting examples of compositions according to the invention and by results of effectiveness.

### EXAMPLES

### Example 1: Active Substance According to the Invention (PA1)

The active substance of example 1 is obtained from a duckweed of the species Spirodela polyrhiza. The active substance of example 1 is obtained by the following method:
Solubilization of at least 50 g/L of Spirodela polyrhiza in water,
Chemical hydrolysis of acid nature and enzymatic hydrolysis
Separation of the soluble and insoluble phases and recovery of the soluble phase,
Heat treatment
Purification and concentration of the soluble phase,
Filtration and sterilizing filtration.

The active substance obtained has the following characteristics:
Content of dry materials=7.4 g/L
Content of Total Neutral Sugars (according to the Dubois method)=2.4 g/L, that is to say 32%, by weight relative to the dry matter
Uronic acid content=3.2 g/L, that is to say 43% by weight relative to the dry matter
Content of apiogalacturonans=55% by weight relative to the dry matter
Galacturonic acid/apiose ratio=3.7
Content of mineral ash=1.7 g/L (23% by weight relative to the dry matter)
pH=3.5
Clear liquid, very pale yellow, faint odor

### Example 2: Active Substance According to the Invention (PA2)

The active substance of example 2 is obtained from a duckweed of the species Spirodela polyrhiza. The active substance of example 2 is obtained by the following method:
Solubilization of at least 50 g/L of Spirodela polyrhiza in water
Chemical hydrolysis of acid nature,
Separation of the soluble and insoluble phases and recovery of the soluble phase
Purification and concentration of the soluble phase
Filtration and sterilizing filtration.

The active substance obtained has the following characteristics:
Content of Dry Materials=6.6 g/L
Content of Total Sugars (according to the Dubois method—glucose range)=2.6 g/L (39% by weight relative to the dry matter)
Uronic acid content=2.2 g/L (33% by weight relative to the dry matter)
Content of apiogalacturonans=40% by weight relative to the dry matter
Galacturonic acid/apiose ratio=2.3
pH=3.5
Clear liquid, light yellow, faint odor

### Example 3: Active Substance Outside the Invention (HI1)

The active substance of example 3 is obtained from fruit from Pyrus malus, comprising saccharides of the homogalacturonan type, or a chain of non-branched galacturonic acid. This active substance is obtained according to the following method:
Solubilization of Pyrus malus fruit in water,
Enzymatic hydrolysis,
Separation of the soluble and insoluble phases,
Heat treatment,
Purification and concentration of the soluble phase
Filtration and sterilizing filtration.

The active substance according to example 3 has the following characteristics:
Content of dry materials=164 g/L
Uronic acid content=126 g/L, that is to say 77% by weight relative to the dry matter
Identification of the sugars present: galacturonic acid
Linked apiose content: 0 g/L
Content of apiogalacturonans=0 g/L
pH=3.1
Clear orange-yellow colored liquid

Despite a polysaccharide structure with a linear chain of galacturonic acids, this active substance differs from the active substance according to the invention in that it does not contain apioses or apiogalacturonans.

### Example 4: Active Substance Outside the Invention (HI2)

The active substance of example 4 is obtained from roots of Ophiopogon japonicus and comprises saccharides of the fructan type, or a fructose chain. This product is obtained according to the following method:
Solubilization of powdered root of Ophiopogon japonicus in water
Enzymatic hydrolysis
Separation of the soluble and insoluble phases
Heat treatment
Purification
Filtration and sterilizing filtration

The product obtained according to example 4 has the following characteristics:
Content of dry materials=123 g/L
Content of total sugars (according to the Dubois method with glucose range)=115 g/L, that is to say 93% by weight relative to the dry matter.
Identification of the sugars present: fructose and glucose
Content of apioses=0 g/L
Content of apiogalacturonans=0 g/L
pH=5.1
Clear yellow-colored liquid

This example differs from the active substance according to the invention in that it does not contain apiogalacturonans.

### Example 5: Composition According to the Invention

An example of a formulation of a serum comprising the active substance according to the invention in gel form is presented in table 1 below.

TABLE 1

| | Ingredients | % |
|---|---|---|
| A | Purified Water | q.s. 100 |
| B | Polyquaternium-37 & Water | 0.70 |
| C | Butylene Glycol | 5.00 |
| | Glyceryl Caprylate | 1.00 |
| | 1,2-Hexanediol | 1.00 |
| | Glycerin | 1.00 |
| | Glycereth-26 | 4.00 |
| | Isopentyldiol | 4.00 |
| | Caprylic/Cupric/Succinic Triglyceride | 1.50 |
| D | INVENTION ACTIVE SUBSTANCE | 3.00 |
| E | Soda 7% | qs pH |

The composition of example 5 can in particular be obtained by the following method:
Sprinkle B into A, stir until a homogeneous gel is obtained.
Add the materials of phase C one-by-one under moderate stirring.
Add D with moderate stirring.
Adjust pH to 6.0-6.5 with E.

The composition is then in the form of a soft, white and shiny opalescent gel having a pH at 1 month equal to 6.2, a viscosity (C/5 rpm) equal to 16,600 cP.

### Example 6: Composition According to the Invention

An example of a formulation comprising the active substance according to the invention in cream form is presented in table 2 below.

TABLE 2

| | Ingredients | % |
|---|---|---|
| A1 | Purified Water | q.s. 100 |
| | Butylene Glycol | 3.00 |
| A2 | Glycerin | 2.00 |
| | Xanthan Gum | 0.15 |
| B | Cetearyl Alcohol & Ceteareth-33 | 4.00 |
| | Myristyl Alcohol & Myristyl Glucoside | 1.50 |
| | C10-18 Triglycerides | 2.00 |
| | Caprylic/Capric Triglyceride | 5.00 |
| | Dicaprylyl Carbonate | 5.00 |
| | Isopropyl Myristate | 4.00 |
| | Ricinus Communis (Castor) Seed Oil | 3.00 |
| | Dimethicone | 1.00 |
| | Tocopherol & Helianthus Annuus | 0.05 |
| | (Sunflower) Seed Oil | |
| C | Sodium Polyacrylate | 1.00 |
| D | INVENTION ACTIVE SUBSTANCE | 3.00 |

The composition of example 6 can in particular be obtained by the following method:
Add A2 into A1 under moderate stirring, stir until a homogeneous gel is obtained and heat to 80° C.
Place B with stirring and heat to 80° C.
Emulsify B in A under shearing stirring for 10 minutes
At 40° C., under moderate stirring, add C then D.

The composition is then in the form of a thick, white and shiny emulsion having a pH at 1 month equal to 6, a viscosity (C/5 rpm) equal to 318,600 cP.

### Example 7: Composition According to the Invention

An example of a formulation comprising the active substance according to the invention in cream form is presented in table 3 below.

TABLE 3

| | Ingredients | % |
|---|---|---|
| A1 | Purified Water | q.s. 100% |
| | Butylene Glycol | 2.00 |
| | Soda Solution 28% | 0.55 |
| A2 | Isopentyldiol | 2.00 |
| | Glycerin | 2.00 |
| | Xanthan Gum | 0.15 |
| B | C20-22 Alkyl Phosphate & C20-22 Alcohols | 3.00 |
| | Cetearyl Alcohol | 1.00 |
| | C10-18 Triglycerides | 2.00 |
| | Squalane | 4.00 |
| | Isononyl Isononanoate | 5.00 |
| | Caprylic/Capric Triglyceride | 7.50 |
| | Isocetyl Stearate | 3.25 |
| | Caprylyl Methicone | 4.00 |
| C | Hydroxyethyl Acrylate/Sodium Acryloyldimethyl | 0.75 |
| | Taurate Copolymer & Polyisobutene & PEG-7 | |
| | Trimethylolpropane Coconut Ether | |
| D | INVENTION ACTIVE SUBSTANCE | 3.00 |

The composition of example 7 can in particular be obtained by the following method:
Add A2 into A1 under moderate stirring, stir until a homogeneous gel is obtained and heat to 80° C.
Place B with stirring and heat to 80° C.
Emulsify B in A under shearing stirring for 10 minutes.
At 40° C., under moderate stirring, add C then D.

The composition is then in the form of a supple, white and shiny emulsion having a pH at 1 month equal to 4.8, a viscosity (C/5 rpm) equal to 58,000 cP.

### Example 8: Composition According to the Invention

An example of a formulation comprising the active substance according to the invention in foundation form is presented in table 4 below.

TABLE 4

| | Ingredients | % |
|---|---|---|
| A1 | Purified Water | q.s. 100% |
| | Sodium Gluconate | 0.20 |
| | Glycerin | 2.00 |
| | Butylene Glycol | 3.00 |
| A2 | Ci 77499 (Iron Oxides) & Silica | 0.10 |
| | Ci 77491 (Iron Oxides) & Silica | 0.50 |
| | Ci 77891 (Titanium Dioxide) & Silica | 6.50 |
| | Ci 77492 (Iron Oxides) & Silica | 1.00 |
| B | Disodium Cetearyl Sulfosuccinate | 1.00 |
| | Glyceryl Stearate | 1.50 |
| | Cetearyl Alcohol | 0.50 |
| | Pentaerythrityl Distearate | 1.20 |
| | Pentaerythrityl Tetrabehenate | 0.80 |
| | Dipentaerythrityl Hexacaprylate/Hexacaprate | 3.00 |
| | Cocoglycerides | 3.00 |
| | Isocetyl Stearate | 3.00 |
| | Propanediol Dicaprylate | 5.00 |
| | Diisopropyl Adipate | 4.00 |
| | Dimethicone | 4.00 |
| | VP/Hexadecene Copolymer | 2.00 |
| | Polyacrylate Crosspolymer-6 | 0.30 |
| | Hydroxyethyl Acrylate/Sodium Acryloyldimethyl | 0.30 |
| | Taurate Copolymer | |
| C | INVENTION ACTIVE SUBSTANCE | 3.00 |

The composition of example 8 can in particular be obtained by the following method:
Disperse A2 in A1 under shearing stirring until homogeneous and heat to 80° C.
Place B with stirring and heat to 80° C.
Emulsify B in A under shearing stirring for 10 minutes.
At 40° C., under moderate stirring, add C.

The composition is then in the form of a thick, pinkish beige emulsion having a pH at 1 month equal to 5.9, a viscosity (C/5 rpm) equal to 123,000 cP.

Mechanism of Action of the Effectiveness of the Active Substance According to the Invention

Test 1—Effect of the Active Substance According to the Invention on the Expression of Aquaporins 3.

The expression of aquaporin 3 (AQP3) was evaluated by quantitative PCR on normal human keratinocytes subjected to water stress.

On D0, human keratinocytes are seeded and incubated at 37° C. in an atmosphere containing 5% CO2. On D4, the cells are treated with the active substance according to the invention at 0.5% (v/v) and then incubated at 37° C. under 5% CO2 in a normal environment or in a dry environment for 24 h. On D5, the cells are recovered and the total RNA extracted. The RNAs were reverse-transcribed and the complementary DNAs obtained were analyzed by the quantitative PCR technique.

The results are presented in table 5 below.

TABLE 5

| | |
|---|---|
| AQP3 Expression | AQP3/dehydrated |
| (%) | control (%) |

Normal keratinocytes

| | |
|---|---|
| Control | 100 |
| ACTIVE SUBSTANCE | 112 |
| ACCORDING TO THE | |
| INVENTION (PA1) 0.5% | |

Dehydrated keratinocytes

| | | |
|---|---|---|
| Control | 80 | |
| ACTIVE SUBSTANCE | 113* | +41 |
| ACCORDING TO THE | | |
| INVENTION (PA1) 0.5% | | |
| ACTIVE SUBSTANCE | 110 | +38 |
| ACCORDING TO THE | | |
| INVENTION (PA1) 0.5% | | |
| EXTRACT OUTSIDE THE | 78 | 0 |
| INVENTION (HI1) 0.025% | | |

The dehydrated model has an expression of aquaporin 3 significantly reduced by 20%. Tested at 0.5% on dehydrated keratinocytes, the active substance according to the invention significantly increases the expression of aquaporin 3 by 41%. The active substance according to the invention thus stimulates the synthesis of one of the key mediators of moisturization.

Test 2—Effect of the Active Substance According to the Invention on the Expression of BGT-1.

The synthesis of BGT-1 was evaluated by immunohistofluorescence on explants of normal human skin subjected to water stress.

For several days, the explants are incubated at 37° C. under 5% CO2 in a normal environment or in a dry environment (water stress). The explants are treated topically with the active substance according to the invention formulated at 0.5% and 1.0% (V/V) or with the placebo.

At the end of the treatments, the explants are recovered and frozen. Sections (4 μm) are then carried out using a cryostat, in order to analyze the synthesis of BGT-1 by immunohistofluorescence using anti-BGT-1 antibodies.

The synthesis of BGT-1 is proportional to the intensity of the fluorescence. Statistical analysis is carried out with the Mann-Whitney non-parametric test.

The results are presented in table 6 below.

TABLE 6

| | |
|---|---|
| | Ability to restore |
| Synthesis of BGT-1 | BGT-1 synthesis |
| (UA) | (%) |

Normal explants

| | |
|---|---|
| Control | 24 |
| ACTIVE SUBSTANCE | 24 |
| ACCORDING TO THE | |
| INVENTION (PA1) 1.0% | |

Dehydrated explants

| | | |
|---|---|---|
| Control | 19 | |
| Placebo | 200^{ns} | |
| ACTIVE SUBSTANCE | 22^{s} | +60 |
| ACCORDING TO THE | | |
| INVENTION (PA1) 0.5% | | |
| ACTIVE SUBSTANCE | 24^{s} | +100 |
| ACCORDING TO THE | | |
| INVENTION (PA1) 1.0% | | |

The dehydrated explant model is characterized by a significant decrease in BGT-1 of 21%. Tested at 1% on a dehydrated model, the active substance according to the invention significantly restores the synthesis of BGT-1. The active substance according to the invention thus makes it possible to restore the osmotic skin equilibrium.

Test 3—Effect of the Active Substance According to the Invention on the Cell Volume.

The volume monitoring of normal human keratinocytes was carried out before and during the application of a hyper-osmotic stress to mimic a dehydration state.

On DO, the keratinocytes are seeded in the culture medium and incubated at 37° C. in an atmosphere containing 5% CO2. They are then treated with the active substance according to the invention at 0.5% (V/V) and incubated at 37° C. in an atmosphere containing 5% CO2. After several days, the keratinocytes are marked with a fluorescent probe, calcein, and then put back into a culture medium containing or not the active substance according to the invention at 0.5% (V/V).

The visualization is then performed using a microscope coupled to a camera and an image analysis system. Cells of the live cells are taken in order to be able to visualize the cells in three dimensions, every two minutes over a period of 18 minutes in total, divided into 2 phases. A 1st phase (0 to 6 minutes) in isoosmotic condition (normal culture medium), and a 2nd phase (8 to 18 minutes) in hyper-osmotic stress.

A quantitative analysis of the cell volume was carried out using an image analysis script developed by the inventor. The results are expressed as percentage of the initial volume of the cells.

The results are presented in table 7 below.

TABLE 7

| | | Cellular volume | | Cellular volume/ |
|---|---|---|---|---|
| | | (%) | | control (%) |
| | | | ACTIVE | ACTIVE |
| | | | SUBSTANCE | SUBSTANCE |
| | | | ACCORDING TO | ACCORDING TO |
| Time | Osmotic | | THE INVENTION | THE INVENTION/ |
| (min) | condition | Control | (PA1) 0.5% | Control |
| 0 | Phase 1: | 100 | 100 | |
| 2 | isoosmotic | 94 | 95 | |
| 4 | condition | 93 | 96 | |
| 6 | | 93 | 95 | |
| 8 | Phase 2. | 58 | 66*** | +14 |
| 10 | hyper- | 58 | 66*** | +14 |
| 12 | osmotic | 58 | 66*** | +14 |
| 14 | stress | 57 | 66*** | +16 |
| 16 | | 57 | 66*** | +16 |
| 18 | | 57 | 65*** | +14 |

After hyper-osmotic stress, the average cell volume is reduced by 43%. When keratinocytes are treated beforehand with the active substance according to the invention at 0.5%, the cell volume after a hyper-osmotic shock is reduced only by 34%. Thus, the active substance according to the invention significantly limits 16% the loss of cell volume following a hyper-osmotic shock. The active substance according to the invention therefore makes it possible to retain the intracellular water.

Test 4—Effect of the Active Substance According to the Invention to Capture Water in the Epidermis.

Test 4 aims to demonstrate the capacity of the active substance according to the invention to capture water within the epidermis, and to compare this efficacy with two examples outside the invention. To do so, the analysis targets the penetration and maintenance of deuterated water (D2O) in the skin 30 minutes and 3 hours after application. The measurements are carried out by Raman microspectroscopy at different depths for 10 minutes.

The exogenous water supply was carried out by application to the skin surface of a patch containing deuterated water (D2O); the marking making it possible to specifically follow its penetration into the skin. This study was carried out on 7 female Caucasian volunteers, aged 45 to 66 years and having dehydrated skin at the forearm.

At T0, the 4 emulsions (placebo emulsion, emulsion containing 3% of the active substance of the invention in example 1, emulsion containing 0.12% of the extract outside the invention according to example 3, emulsion containing 0.12% of the extract outside the invention according to example 4) are massaged onto the volunteers until complete penetration is achieved. At T3 hours, an occlusive patch containing D2O is applied for 30 minutes, on the zones previously treated with the emulsions. After 30 minutes, the patch is removed and the residual water is removed. The Raman microspectroscopy measurements are carried out.

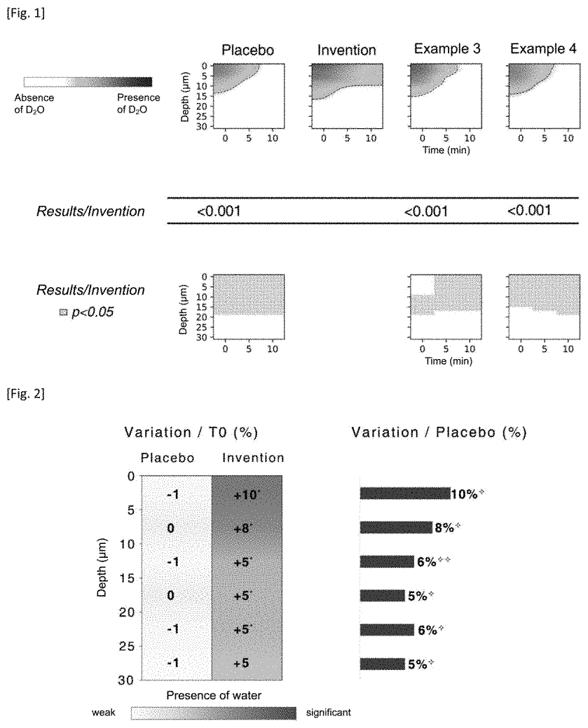

The results on the amount of D2O in the epidermis for the 4 emulsions are shown in FIG. 1.

In comparison to the placebo and to the extracts outside the invention, examples 3 and 4, the active substance according to the invention has a significantly different effect. Water enters in a larger amount, goes deeper, and remains longer in the skin. These results thus reflect in vivo the higher hygroscopic power of the active substance according to the invention relative to the extracts outside the invention.

In vivo evaluation of the biological activity of the active substance according to the invention

Test 5—Study of the Total Water Content in the Epidermis.

The study aims to evaluate in vivo the immediate effect and the long-term effect of the active substance according to the invention formulated at 3% in an emulsion on the total water content of the epidermis in comparison to a placebo formula.

The immediate effect of the active substance according to the invention was studied in 19 healthy Caucasian volunteers, female, aged 35 to 65 years, having dehydrated skin on their faces, an irregular microrelief at the crow's feet, and a dull complexion.

The long-term effect of the active substance according to the invention was studied in 17 healthy Caucasian volunteers, female, aged 44 to 65 years, having dehydrated skin on their faces, an irregular microrelief at the crow's feet, and a dull complexion.

The measurements are carried out by Raman microspectroscopy on symmetrical zones at the cheeks, before and then 3 hours after a single application of the products being studied on half the face (immediate effect), and before and after 21 and 42 days of twice-daily treatment on a half-face (long-term effect). The total water content was evaluated by studying the ratio of the Raman vOH-total/vCH bands.

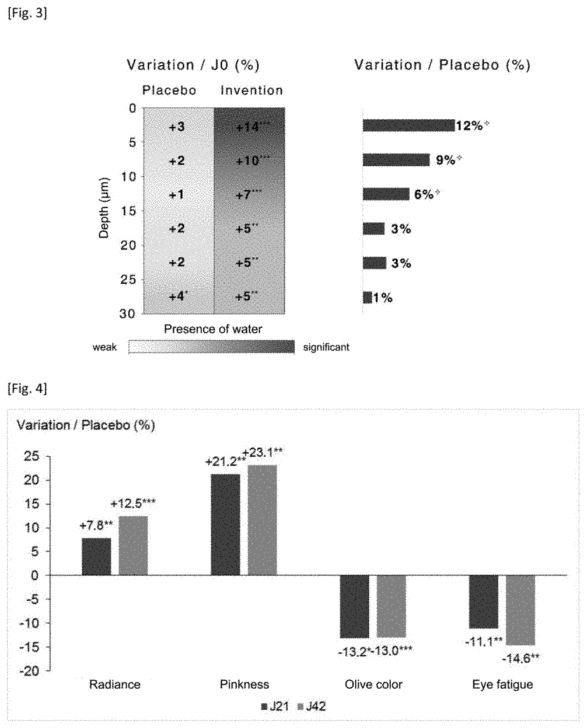

The results corresponding to the effect of the active substance according to the invention, formulated at 3% in an emulsion, on the total amount of water at the epidermis of Caucasian volunteers are presented in FIG. 2 for the immediate effect and FIG. 3 for the long-term effect.

From 3 hours after a single application, the active substance according to the invention formulated at 3% in emulsion form significantly increases the total amount of water retained in the epidermis. These increases were observed in 60% of the volunteers. This effect intensifies after 42 days of twice-daily application. Indeed, the active substance according to the invention causes a significant increase in the total amount of water in the stratum corneum. These increases were observed in over 75% of the volunteers.

Test 6—Study of Natural Moisturizing Factors.

The objective of this study is to evaluate in vivo, in comparison to a placebo formula, the effect of the active substance according to the invention formulated at 3% in emulsion on the natural moisturizing factors, a set of intracorneocytic hydrophilic substances (e.g. lactate, urea, inorganic ion, amino acid) present in the stratum corneum.

The study was carried out on two groups of healthy female Caucasian volunteers who had dehydrated skin on their faces, an irregular microrelief at the crow's feet, and a dull complexion. A placebo group consisting of 18 subjects aged 35 to 67 years and a group of the active substance according to the invention consisting of 20 subjects aged 36 to 67 years old. The volunteers applied the active substance according to the invention or the placebo, over the whole of the face, twice daily, for 42 days. The lactate was assayed by fluorimetry on the basis of samples carried out at the cheeks.

The results corresponding to the effect of the active substance according to the invention formulated at 3% in emulsion form on lactate concentration are presented in table 8.

TABLE 8

| | Variation | Variation |
|---|---|---|
| | D 21/D 0 | D 42/D 0 |
| PLACEBO | −1.0^{ns} | −0.9^{ns} |
| ACTIVE SUBSTANCE ACCORDING | +31.7^{s} | +50.2^{s} |
| TO THE INVENTION (PA1) 3% | | |
| Comparison of ACTIVE SUBSTANCE | +32.7% | +51.2% |
| ACCORDING TO THE INVENTION/ | | |
| Placebo | | |

Tested at 3% on a Caucasian panel, the active substance according to the invention significantly increases the lactate concentration compared to the placebo. Indeed, after 21 days of twice-daily treatment, the lactate concentration is significantly increased by 32.7%. This effect intensifies after 42 days of treatment with a significant increase of 51.2%.

Test 7—Study of Water Losses

The objective of this study is to evaluate in vivo the effect of the active substance according to the invention formulated at 3% in emulsion form on the water losses after a single application, in comparison with a placebo formula. The study was carried out on 16 healthy female Caucasian volunteers aged 35 to 65 years, having dehydrated skin on their faces, an irregular microrelief at the crow's feet, and a dull complexion. The measurements were carried out using a Tewameter® (Courage & Khazaka) on symmetrical areas at the cheeks, before then 3 and 6 hours after a single application of the products in half-face.

The results corresponding to the capacity of the active substance according to the invention formulated at 3% in emulsion form to limit water losses are presented in table 9.

TABLE 9

| | Variation | Variation |
|---|---|---|
| | T3 H/T0 | T6 H/T0 |
| PLACEBO | +0.4^{ns} | +2.6^{ns} |
| ACTIVE SUBSTANCE | −11.5^{s} | −13.1^{s} |
| ACCORDING TO THE INVENTION | | |
| (PA1) 3% | | |
| Comparison of ACTIVE | −11.9% | −15.7% |
| SUBSTANCE ACCORDING TO | | |
| THE INVENTION/Placebo | | |

From 3 hours after a single application, the active substance according to the invention formulated at 3% makes it possible to reduce the water losses by 11.9% compared to the placebo. This action amplifies 6 hours after application, with a 15.7% reduction in water losses, the effect observed in 69% of the subjects. The active substance according to the invention therefore makes it possible to retain water within the epidermis.

Evaluation of the Cosmetic Efficacy of the Active Substance According to the Invention

Test 8—Direct Moisturizing Effect of the Active Substance According to the Invention

The objective of this study is to evaluate in vivo the immediate effect of the active substance according to the invention, formulated at 3% in emulsion form, on the hydration of the deep and surface layers of the skin of Caucasian volunteers compared to a placebo formula. This study was carried out on 16 healthy female Caucasian volunteers aged 35 to 65 years, having dehydrated skin on their faces, an irregular microrelief at the crow's feet, and a dull complexion. The immediate moisturizing effect on the epidermis and the upper dermis was evaluated by measuring the hydration level using a MoistureMeter-D (Delfin Technologies). The ability of the active substance according to the invention to immediately moisturize the stratum corneum was evaluated via the measurement of hydration level using a Corneometer® CM825 (Courage & Khazaka). The measurements were carried out 3 and 6 hours after a single application of the active substance according to the invention and the placebo to a half-face.

The results that show the immediate effect of the active substance according to the invention formulated at 3% in emulsion form, on the hydration of the epidermis and of the upper dermis is presented in table 10 and on the hydration of the stratum corneum in table 11.

TABLE 10

| | Variation | Variation |
|---|---|---|
| On the epidermis and the upper dermis | T3 H/T0 | T6 H/T0 |
| PLACEBO | −1.5^{ns} | −1.6^{ns} |
| ACTIVE SUBSTANCE ACCORDING | +10.4^{s} | +14.2^{s} |
| TO THE INVENTION (PA1) 3% | | |
| Comparison of ACTIVE SUBSTANCE | +11.9% | +15.8% |
| ACCORDING TO THE INVENTION/ | | |
| Placebo | | |

From 3 hours after a single application, in comparison to the placebo, the active substance according to the invention formulated at 3% in emulsion form significantly increases the hydration of the skin in the epidermis and the upper dermis by 11.9%. This effect extends 6 hours after the application with an increase in hydration equal to 15.8%. These results were observed in 63% and 81% of the subjects.

TABLE 11

| | Variation | Variation |
|---|---|---|
| On the Stratum corneal | T3 H/T0 | T6 H/T0 |
| PLACEBO | +0.4^{ns} | +4.7^{ns} |
| ACTIVE SUBSTANCE ACCORDING | +18.2^{s} | −29.8^{s} |
| TO THE INVENTION (PA1) 3% | | |
| Comparison of ACTIVE SUBSTANCE | +17.8% | +25.1% |
| ACCORDING TO THE INVENTION/ | | |
| Placebo | | |

the active substance according to the invention immediately improves surface hydration in volunteers. Indeed, from 3 hours after a single application, the active substance according to the invention formulated at 3% in emulsion increases the hydration of the stratum corneum by 17.8%. This action extends and intensifies 6 hours after treatment with a significant improvement in surface hydration, by 25.1%. This effect is observed in 88% of the volunteers.

Thus, the active substance according to the invention formulated at 3% in emulsion improves the immediate hydration of the upper dermis up to the stratum corneum from 3 hours after a single application in Caucasian volunteers.

Test 9—Long-Term Moisturizing Effect of the Active Substance According to the Invention

The objective of this study is to evaluate the long-term effect of the active substance according to the invention on the hydration of the deep and surface layers of the epidermis of Caucasian and Asian volunteers, in comparison to a placebo formula.

This study was carried out on a Caucasian panel of 17 healthy Caucasian volunteers, female, ages 44 to 65, and on an Asian panel of 63 healthy volunteers, female, ages 41 to 65, having dehydrated skin on their faces, an irregular microrelief at the crow's feet, and a dull complexion.

The deep hydration was evaluated by measuring the hydration level using a MoistureMeter-D and surface hydration via the measurement of hydration level using a Corneometer® CM825.

The Caucasian volunteers applied the active substance according to the invention formulated at 3% in emulsion form and the placebo onto a half-face, twice-daily for 21 and 42 days.

The Asian volunteers were divided into 2 groups who respectively applied to their whole face the formula containing the active substance according to the invention at 3% or the placebo formula twice-daily for 21 and 42 days. The group receiving the active substance according to the invention consisted of 32 volunteers and the group receiving the placebo of 31 volunteers.

The results showing the long-term effect on the hydration of the epidermis and the upper dermis and on the surface hydration of Caucasian volunteers are presented in tables 12 and 13, respectively. The results showing the long-term effect on the surface hydration of Asian volunteers are presented in table 14.

TABLE 12

| Hydration of the epidermis and of the | Variation | Variation |
|---|---|---|
| upper dermis Caucasian panel | D 21/D 0 | D 42/D 0 |
| PLACEBO | +10.4^{s} | +2.4^{ns} |
| ACTIVE SUBSTANCE ACCORDING | +21.0^{s} | +18.0^{s} |
| TO THE INVENTION (PA1) 3% | | |
| Comparison of ACTIVE SUBSTANCE | +10.6% | +15.5% |
| ACCORDING TO THE INVENTION/ | | |
| Placebo | | |

After 21 days of treatment, the active substance according to the invention significantly increases the hydration of the epidermis and the upper dermis by 10.6%. This effect intensifies after 42 days of treatment with a significant increase of 15.5%. This effect is observed in 88% of the volunteers.

TABLE 13

| Hydration of the stratum corneum | Variation | Variation |
|---|---|---|
| Caucasian panel | D 21/D 0 | D 42/D 0 |
| PLACEBO | +4.5^{ns} | +2.8^{ns} |
| ACTIVE SUBSTANCE ACCORDING | +10.5^{s} | +12.8^{s} |
| TO THE INVENTION (PA1) 3% | | |
| Comparison of ACTIVE SUBSTANCE | +5.9% | +10.0% |
| ACCORDING TO THE INVENTION/ | | |
| Placebo | | |

After 21 days of treatment, the active substance according to the invention significantly increases the hydration of the stratum corneum of Caucasian volunteers by 5.9%. This effect intensifies after 42 days of treatment with a significant improvement of 10.0%. This effect is observed in 76% of the volunteers.

TABLE 14

| Hydration of the stratum corneum | Variation | Variation |
|---|---|---|
| Asian panel | D 21/D 0 | D 42/D 0 |
| PLACEBO | −0.5^{s} | −1.9^{ns} |
| ACTIVE SUBSTANCE ACCORDING | +12.4^{s} | +20.6^{s} |
| TO THE INVENTION (PA1) 3% | | |
| Comparison of ACTIVE SUBSTANCE | +12.9% | +22.5% |
| ACCORDING TO THE INVENTION/ | | |
| Placebo | | |

After 21 days of treatment, the active substance according to the invention significantly increases the hydration of the stratum corneum of Asian volunteers by 12.9%. This effect intensifies after 42 days of treatment with a significant increase of 22.5%. This effect is observed in 97% of the volunteers.

Thus, the active substance according to the invention formulated at 3% in emulsion form improves the hydration of the surface layers of the epidermis to the upper dermis after 6 weeks of twice-daily treatment in Caucasian and Asian volunteers.

Test 10—Plumping Effect of the Active Substance According to the Invention

The objective of this study is to evaluate in vivo the plumping effect of the active substance according to the invention formulated at 3% in emulsion form, compared to a placebo formula on Caucasian volunteers. The panel is composed of two groups of healthy volunteers who had dehydrated skin on their faces, an irregular microrelief at the crow's feet, and a dull complexion. A 1st group treated with the active substance according to the invention comprises 20 volunteers aged 36 to 67 years and a 2^{nd} group treated with the placebo comprises 18 volunteers aged from 35 to 67 years old. The plumping effect was studied by measuring the volumetry of the face (EvaFACE^{3D}-S5 system, Eotech) after 21 and 42 days of twice-daily application of the active substance according to the invention or the placebo on the whole face.

The results corresponding to the effect of the active substance according to the invention formulated at 3% in emulsion form on the positive volume parameter are presented in table 15.

TABLE 15

| | Variation | Variation |
|---|---|---|
| | D 21/D 0 | D 42/D 0 |
| PLACEBO | −0.8^{s} | −3.6^{ns} |
| ACTIVE SUBSTANCE ACCORDING | +31.2^{s} | +43.7^{s} |
| TO THE INVENTION (PA1) 3% | | |
| Comparison of ACTIVE SUBSTANCE | +32.0% | +47.3% |
| ACCORDING TO THE INVENTION/ | | |
| Placebo | | |

Thus, the active substance according to the invention, compared to the placebo, significantly plumps the skin of the face of Caucasian volunteers. Indeed, after 21 days of twice-daily application throughout the face, the active substance according to the invention formulated at 3% in emulsion form significantly improves the positive volume parameter, which is characteristic of a plumping effect, by 32.0%. This effect was observed in 82% of the volunteers. This effect extends and intensifies after 42 days of treatment, at which point the increase in the positive volume parameter is equal to 47.3%. This increase is visible in 67% of the volunteers.

Test 11—Smoothing Effect of the Active Substance According to the Invention

The objective of this study is to evaluate in vivo the smoothing effect of the active substance according to the invention formulated at 3% in emulsion form, compared to a placebo formula on Asian volunteers. The panel was composed of two groups of healthy volunteers who had dehydrated skin on their faces, an irregular microrelief at the crow's feet and under the eyes, and a dull complexion. A first group receiving the active substance according to the invention comprises 32 volunteers aged 43 to 64 and a 2^{nd} group receiving the placebo comprises 31 volunteers aged 41 to 65.

The smoothing effect was evaluated by clinical scoring by a dermatologist before and after applying the active substance according to the invention or the placebo on the whole face, twice-daily for 42 days.

The results corresponding to the smoothing effect, evaluated by clinical scoring of the area below the eye, of the active substance according to the invention, formulated at 3% in emulsion form, are presented in table 16.

TABLE 16

| | Variation | Variation |
|---|---|---|
| | D 21/D 0 | D 42/D 0 |
| PLACEBO | −1.6^{s} | −4.0^{ns} |
| ACTIVE SUBSTANCE ACCORDING | −5.6^{s} | −11.4^{s} |
| TO THE INVENTION (PA1) 3% | | |
| Comparison of ACTIVE SUBSTANCE | −4.0% | −7.4% |
| ACCORDING TO THE INVENTION/ | | |
| Placebo | | |

The active substance according to the invention, in comparison to the placebo, has a significant smoothing effect on the area of the contour of the eye in Asian volunteers. Indeed, after 21 days of twice-daily application over the whole of the face, the active substance according to the invention formulated at 3% in emulsion form, significantly reduces by 4.0% the stage of wrinkles under the eyes. These effects extend after 42 days of treatment with a reduction in the wrinkles under the eyes of 7.4%. This effect is observed in 66% of the volunteers.

Test 12—Effect of the Active Substance According to the Invention on Complexion Radiance.

The objective of this study is to evaluate in vivo what effect the active substance according to the invention formulated at 3% in emulsion form has on complexion radiance, in comparison to a placebo formula, on Caucasian and Asian volunteers, after 21 and 42 days of twice-daily application of the active substance according to the invention or of the placebo onto the whole face.

The Caucasian and Asian panels were composed of two groups of healthy volunteers who had dehydrated skin on their faces, an irregular microrelief at the crow's feet and under the eyes, and a dull complexion.

In a Caucasian panel, a 1^{st} group receiving the active substance according to the invention was formed of 20 volunteers aged 36 to 67 and a 2^{nd} group receiving the placebo was formed of 18 volunteers aged 35 to 67.

In an Asian panel, a 1^{st} group receiving the active substance according to the invention was formed of 32 volunteers aged 43 to 64 and a 2^{nd} group receiving the placebo was formed of 31 volunteers aged 41 to 65.

Complexion radiance was evaluated by clinical scoring by two experts trained (Caucasian panel) or a dermatologist (Asian panel), before and after 21 and 42 days of twice-daily application of the active substance according to the invention or the placebo on the whole face.

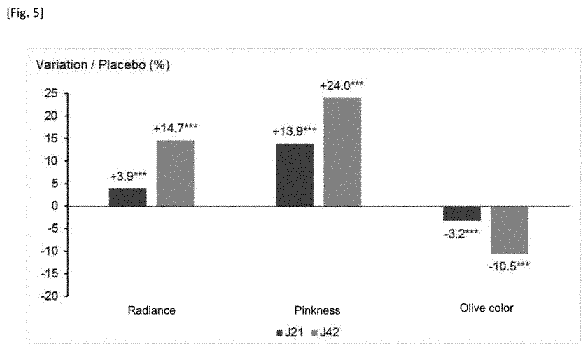

The results corresponding to the effect of the active substance according to the invention formulated at 3% in emulsion on complexion radiance for the Caucasian and Asian panel are presented in FIGS. 4 and 5, respectively.

FIG. 4 shows that the active substance according to the invention significantly improves the characteristic parameters of the complexion radiance of Caucasian volunteers. Indeed, from 21 days of applications, the active substance according to the invention formulated at 3% in emulsion form makes the complexion more luminous and fresher by significantly increasing skin radiance by 7.8% and pinkness by 21.2%. It significantly reduces olive color by 13.2% and eye fatigue by 11.1%. These actions extend after 42 days of treatment with a 12.5% increase in skin radiance and 23.1% increase in pinkness. A 13.0% decrease in olive color and 14.6% decrease in eye fatigue in Caucasian volunteers is also observed.

FIG. 5 shows that the active substance according to the invention significantly improves the characteristic parameters of the complexion radiance of Asian volunteers. Indeed, from 21 days of applications, the active substance according to the invention formulated at 3% in emulsion form makes the complexion more luminous and fresher by significantly increasing skin radiance by 3.9% and pinkness by 13.9%. It significantly reduces olive color by 3.2%. These effects are extended after 42 days of treatment, with a 14.7% increase in skin radiance and 24% increase in pinkness. A 10.5% decrease in olive color is also observed in the Asian volunteers.

Thus, the active substance according to the invention formulated at 3% in emulsion improves the complexion radiance of Caucasian and Asian volunteers.

1. A cosmetic method for a pluming effect, the cosmetic method comprising: applying a cosmetic active substance comprising at least one extract of Spirodela polyrhiza comprising apiogalacturonans.

2. The cosmetic method of claim 1, wherein the active substance also has a moisturizing effect and/or improves the radiance of the complexion.

3. The cosmetic method of claim 1, wherein the active substance increases the cutaneous cell volume, and/or the expression of aquaporin 3 and/or the synthesis of the BGT-1 transporter.

4. The cosmetic method of claim 1, wherein the active substance comprises at least one extract of Spirodela polyrhiza, and wherein the extract is a hydrolysate.

5. The cosmetic method of claim 4, wherein the extract has a hygroscopic potential greater than or equal to 15 molecules of water interacting by monomer.

6. The cosmetic method of claim 1, wherein the active substance comprises apiogalacturonans in an amount at least 40% by weight of the dry matter of the extract.

7. The cosmetic method of claim 4, wherein the extract comprises carbohydrates representing at least 70% by weight of the dry matter of the extract.

8. The cosmetic method of claim 4, wherein the extract has a galacturonic acid/apiose ratio of between 1 and 5.

9. The cosmetic method of claim 4, wherein the extract is obtainable by a method comprising the following steps:
a. Solubilization of at least 50 g/L of Spirodela polyrhiza in water,
b. Chemical hydrolysis of acid nature, or chemical hydrolysis of acid nature and enzymatic hydrolysis,
c. Separation of the soluble and insoluble phases and recovery of the soluble phase,
d. Purification and concentration of the soluble phase,
e. Filtration and sterilizing filtration.

10. The cosmetic method of claim 4, wherein the cosmetic method comprises applying topically the active substance formulated as a composition comprising the active substance and a physiologically acceptable medium.

11. The cosmetic method of claim 10, wherein the active substance represents at least 0.1% by weight of the total weight of the composition.

12. (canceled)
